(19) United States
(12) Patent Application Publication
(10) Pub. No.: US 2009/0299387 A1
(43) Pub. Date: Dec. 3, 2009

(54) METHOD AND APPARATUS FOR FLUIDLY ISOLATING A PORTION OF A BODY LUMEN WALL FROM FLOW THROUGH THE BODY LUMEN

(75) Inventor: Jose Luis Navia, Shaker Heights, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/475,475

(22) Filed: May 30, 2009

Related U.S. Application Data
(60) Provisional application No. 61/058,252, filed on Jun. 3, 2008.

(51) Int. Cl. A61B 17/11

(52) U.S. Cl. 606/153

(57) ABSTRACT

An apparatus for fluidly isolating a portion of a blood vessel wall from bloodflow at an anastomosis site within a blood vessel is described. An insertion catheter has longitudinally spaced proximal and distal catheter ends and an operative lumen extending therebetween, and is configured for insertion into the blood vessel at an insertion location spaced apart from the anastomosis site. An isolation device is attached to the distal catheter end, and includes a concave working surface bounded by an isolation rim and a bloodflow surface opposite the working surface. The isolation rim is configured to contact at least a portion of the blood vessel longitudinally aligned with, and radially spaced from, the anastomosis site. The bloodflow surface is in contact with bloodflow past the anastomosis site within the blood vessel when the isolation device engages the blood vessel wall. A retention means is attached to the isolation device and in fluid communication with the operative lumen. The retention means is configured to exert force on the blood vessel wall to at least partially engage the blood vessel wall with the isolation device. A method of fluidly isolating a portion of a blood vessel wall from bloodflow at an anastomosis site within a blood vessel is also provided.

### RELATED APPLICATION

This application claims priority from U.S. Provisional Patent Application Ser. No. 61/058,252, filed Jun. 3, 2008, the subject matter of which is incorporated herein by reference.

### TECHNICAL FIELD

The present invention relates to an apparatus and method for fluidly isolating a portion of a body lumen wall from flow through the body lumen and, more particularly, to an apparatus and method for fluidly isolating an anastomosis site on a blood vessel wall from bloodflow through the vessel.

### BACKGROUND OF THE INVENTION

Many different types of surgeries require the creation of an anastomosis between two body lumens to allow fluid flow therebetween. For example, during a coronary artery bypass grafting surgery, an end-to-side anastomosis may be used to place a native blood vessel into fluid communication with a graft vessel, hence facilitating bypass bloodflow through the graft vessel to or from the native blood vessel.

Because bloodflow through the native vessel is difficult to contain within the native vessel during the anastomosis procedure, it is often challenging for a surgeon to readily and efficiently create a fluidtight anastomosis link between the native and graft blood vessels. In addition to the detrimental results of loss of blood volume, uncontained bloodflow can visually obscure the operating field, cause thromboses, drain into other body areas in an unwanted manner, and complicate placement and positioning of the graft vessel and other items within the operating field.

Currently, surgeons often choose to block bloodflow through the native blood vessel during the anastomosis procedure by placing one or more clamps on the outside of the vessel near the anastomosis site, to thus address certain of the aforementioned conditions. However, prevention of bloodflow via clamping can cause thromboses (which can be swept along into other portions of the patient's vascular system once bloodflow is resumed), cause vessel dissection, cause tissue ischemia downstream from the clamping site, and/or dislodge particulate material from the native blood vessel walls, which may cause adverse side effects from the anastomosis creation, as well. In addition, it is not possible to safely clamp calcified, and thus relatively brittle, blood vessels.

### SUMMARY OF THE INVENTION

In an embodiment of the present invention, an apparatus for fluidly isolating a portion of a blood vessel wall from bloodflow at an anastomosis site within a blood vessel is described. An insertion catheter has longitudinally spaced proximal and distal catheter ends and an operative lumen extending therebetween, and is configured for insertion into the blood vessel at an insertion location spaced apart from the anastomosis site. An isolation device is attached to the distal catheter end, and includes a concave working surface bounded by an isolation rim and a bloodflow surface opposite the working surface. The isolation rim is configured to contact at least a portion of the blood vessel longitudinally aligned with, and radially spaced from, the anastomosis site. The bloodflow surface is in contact with bloodflow past the anastomosis site within the blood vessel when the isolation device engages the blood vessel wall. A retention means is attached to the isolation device and in fluid communication with the operative lumen. The retention means is configured to exert force on the blood vessel wall to at least partially engage the blood vessel wall with the isolation device.

In an embodiment of the present invention, an anastomosis system is described. Means are provided for fluidly isolating an anastomosis site on a blood vessel wall from bloodflow through the blood vessel. Means are provided for inserting the means for fluidly isolating into the blood vessel at a location spaced apart from the anastomosis site. Means are provided for retaining the means for fluidly isolating at the anastomosis site. When the means for retaining is engaged, the means for fluidly isolating contacts at least a portion of the blood vessel longitudinally aligned with, and radially spaced from, the anastomosis site.

In an embodiment of the present invention, a method for fluidly isolating a portion of a blood vessel wall from bloodflow at an anastomosis site within a blood vessel is described. An insertion catheter having longitudinally spaced proximal and distal catheter ends and an operative lumen extending therebetween is provided. An isolation device attached to the distal catheter end is provided. The isolation device includes a concave working surface bounded by an isolation rim and a bloodflow surface opposite the working surface. The distal catheter end is inserted into the blood vessel at an insertion location spaced apart from the anastomosis site. At least a portion of the blood vessel longitudinally aligned with, and radially spaced from, the anastomosis site is contacted with the isolation rim. The bloodflow surface is contacted with bloodflow past the anastomosis site within the blood vessel. Force is exerted on the blood vessel wall to at least partially engage the blood vessel wall with the isolation device.

### BRIEF DESCRIPTION OF THE DRAWINGS

For a better understanding of the invention, reference may be made to the accompanying drawings, in which:

FIG. 1 is a top view of a first embodiment of the present invention;

FIG. 2 is a side view taken along line 2-2 of FIG. 1;

FIG. 3 is a schematic partial side view of the embodiment of FIG. 1 in an example use environment;

FIG. 4 is a cross-sectional view taken along line 4-4 of FIG. 3;

FIG. 5 is a top view of the embodiment of FIG. 1 in an example use environment;

FIG. 6 is a side perspective view of a second embodiment of the present invention;

FIG. 7 is a schematic partial side view of the embodiment of FIG. 6 in an example use environment;

FIG. 8 is a perspective cross-sectional view taken along line 8-8 of FIG. 7;

FIG. 9 is a side view of the embodiment of FIG. 6 in a first example use environment;

FIG. 10 is a cross-sectional view of the embodiment of FIG. 6 in a second example use environment; and

FIG. 11 is a partial cross-sectional view of the embodiment of FIG. 6 in a third example use embodiment.

### DESCRIPTION OF EMBODIMENTS

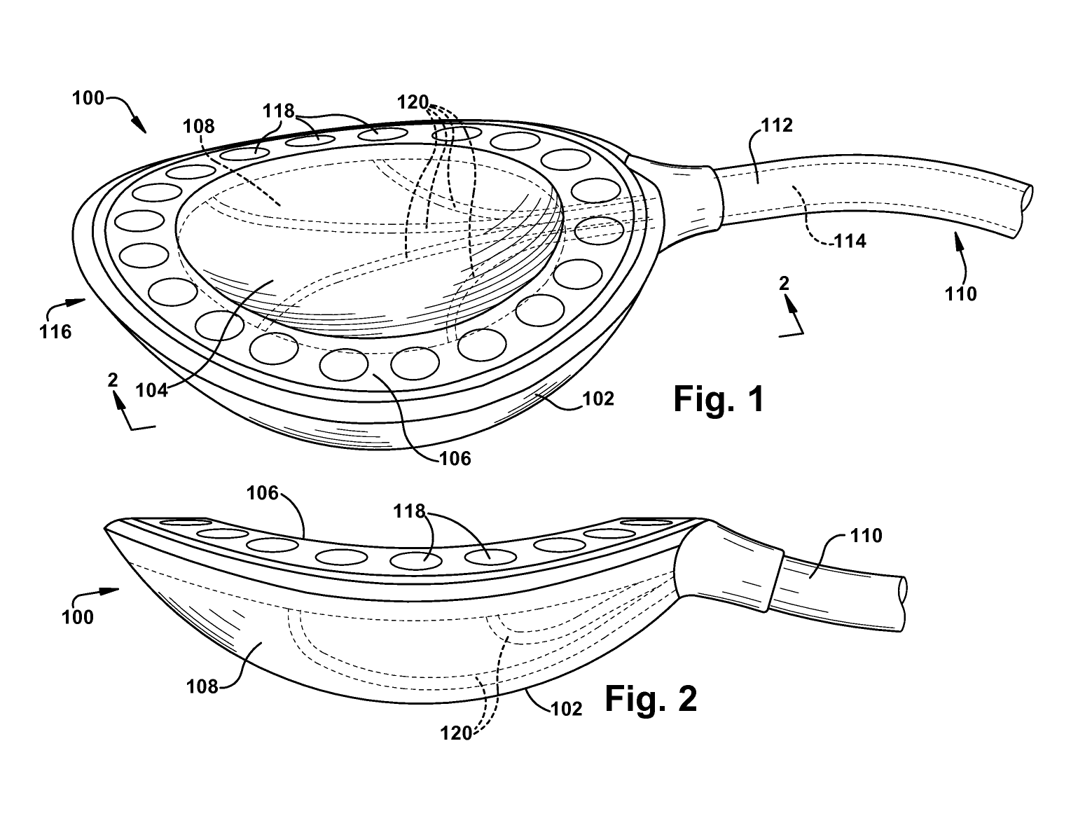

In accordance with a first example embodiment of the present invention, FIG. 1 depicts an apparatus 100 for fluidly isolating a portion of a blood vessel wall from bloodflow at an anastomosis site within a blood vessel. The apparatus 100 is a component of a system which may be used to isolate a portion of any body lumen wall from a concurrently continuing fluid flow within that body lumen, for any reason. Here, the apparatus 100 will be shown and described as an anastomosis apparatus, but the apparatus could be used in any suitable application, such as in any suitable body lumen (e.g., the carotid, aortic, or any other artery; the vena cava, femoral, or any other vein; the esophagus, large or small intestine, or any other non-cardiovascular lumen; any body organ; or any other desired anastomosis site). Also for ease of description, the body lumens in the below description will be referenced as being native and graft blood vessels, with blood flowing through the native vessel during the anastomosis procedure. However, the present invention is not limited in form, application, or functionality by such description. For example, two or more native vessels, grafts, stents, and other body lumens, or any other structures as desired, could be placed into any desired anastomosis relationship using any embodiment of the present invention.

The apparatus 100 represents one possible means for fluidly isolating an anastomosis site on a blood vessel wall from bloodflow through the blood vessel to provide a substantially bloodflow-free operating field for creation of the anastomosis connection between the native and graft blood vessels, while allowing maintenance of bloodflow through the native blood vessel and downstream of the anastomosis site access wound. In the first embodiment, shown in FIGS. 1-5, an isolation device 102 includes a concave working surface 104 bounded by an isolation rim 106 and a bloodflow surface 108 opposite the working surface. The isolation rim 106 is configured to contact at least a portion of a blood vessel longitudinally aligned with, and radially spaced from, the anastomosis site, as shown and discussed below. The bloodflow surface 108 is in contact with bloodflow past the anastomosis site within the blood vessel when the isolation device 102 engages the blood vessel wall, as will be discussed in detail below.

The anastomosis system also includes a component providing means for inserting the apparatus 100 into the blood vessel at a location spaced apart from the anastomosis site. For example, as shown in FIG. 1, an insertion catheter 110 has longitudinally spaced proximal catheter end (not shown) and distal catheter end 112. An operative lumen 114 may extend between the proximal and distal catheter ends 112. The isolation device 102 is attached to the distal catheter end 112 of the catheter 110.

At least a portion of the isolation device 102 may be selectively collapsible for insertion to and/or removal from the blood vessel.

The catheter 110 can be inserted into the blood vessel at any suitable location, but is preferably inserted into the blood vessel at an insertion location spaced apart from the anastomosis site. For example, the distal catheter end 112 can be percutaneously inserted in a known manner (e.g., a Seldinger or open insertion procedure) into a relatively remote blood vessel of the body (e.g., a femoral or jugular vessel) and then advanced through the vascular system to the anastomosis site. As another example, the catheter 110 could be inserted through the skin near the anastomosis area using a known minimally invasive technique, and then inserted into the blood vessel through a puncture spaced at least slightly apart from the anastomosis site. The catheter 110 can include one or more guidewires, working and/or visualization lumens, or the like (not shown).

One of ordinary skill in the art can readily select an insertion site and/or method responsive to a desired stability property of the isolation device 102. That is, if a puncture near the anastomosis site would enable better stabilization of the isolation device 102 than would a remote insertion, the former may be chosen for the insertion site. Likewise, the insertion site and/or method could be chosen responsive to a structural, material, chemical, and/or any other physical property of the isolation device 102 or of the body structures at or near the anastomosis site, or encountered by the isolation device en route to the anastomosis site.

The anastomosis system also includes means for retaining the isolation device 102 at the anastomosis site. For example, retention means 116 may be attached to the isolation device 102 and be configured to exert force on the blood vessel wall to at least partially engage the blood vessel wall with the isolation device. “Engage” is used herein to indicate a relationship in which sufficient force (frictional or otherwise) is exerted between two structures to substantially prevent relative movement between the structures under most operating conditions, although there is not necessarily an interlocking relationship formed between two “engaged” structures. The retention means may be attached to the isolation device 102 and are optionally in fluid communication with the operative lumen 114 of the catheter 110, for reasons that will be made clear shortly.

The first embodiment of the present invention will now be discussed in further detail, with reference to FIGS. 1-5. As seen in FIG. 1, the retention means 116 of the isolation device 102 of the first embodiment includes at least one vacuum port 118 located on the isolation rim 106. The vacuum ports 118 are in fluid connection with a vacuum source (not shown) outside the patient's body via the operative lumen 114 of the catheter 110. At least one vacuum line 120, a plurality of which are depicted as being at least partially located between the working and bloodflow surfaces 104 and 108 in the Figures, may be used to link one or more vacuum ports 118 with the operative lumen 114. FIG. 2 is a side view of the apparatus 100, in which the vacuum lines 120 are shown in phantom line as extending within the bloodflow surface 108 from the catheter 110 to different locations adjacent the isolation rim 106. Though the vacuum lines 120 are depicted as being tubes sandwiched between the working and bloodflow surfaces 104 and 108, the vacuum lines 120 may be located at, or extend through or past, any other suitable location on or near the isolation device 102. Additionally, the vacuum lines 120 may be separately provided, or may be formed integrally with one or more other components of the isolation device 102. For example, the isolation device 102 could include a one-piece molding including the working and bloodflow surfaces 104 and 108 and the vacuum lines 120.

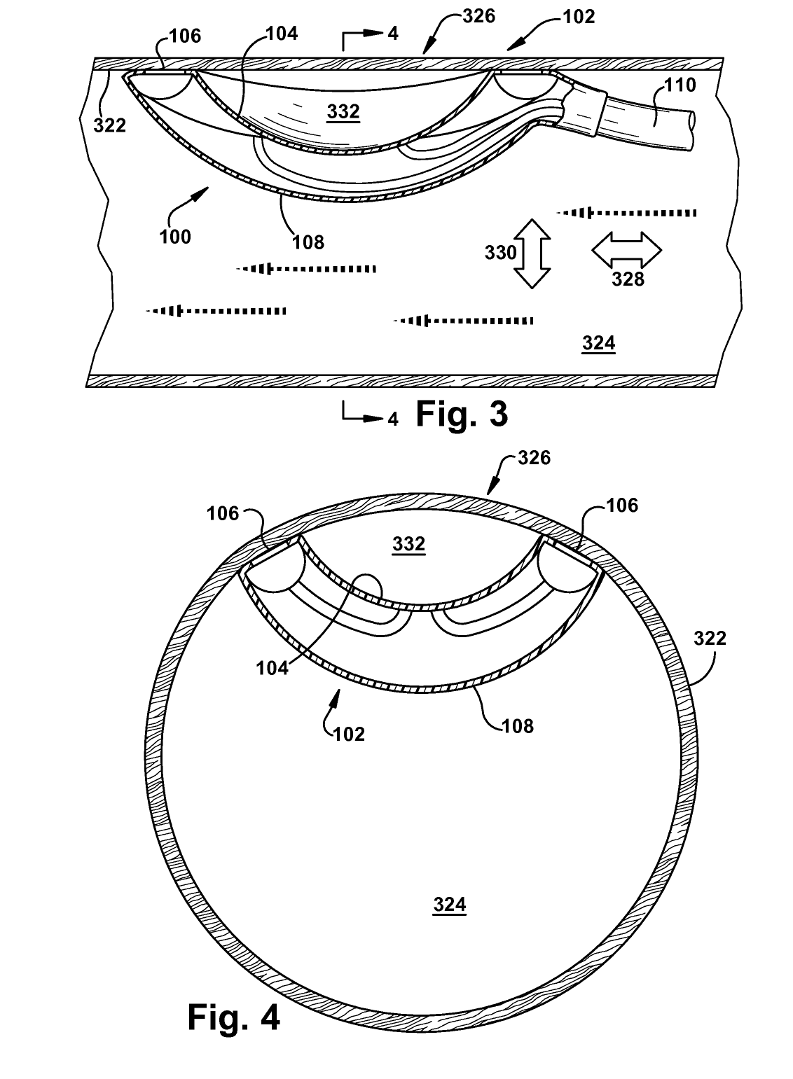

The vacuum ports 118 exert an inward tension force to at least partially engage the blood vessel wall 322 with the isolation device 102, as shown schematically in FIG. 3. In the view of FIG. 3, the apparatus 100 has been inserted into the blood vessel 324 at a location (not shown) spaced apart from the anastomosis site 326 and moved longitudinally (in the orientation indicated by longitudinal arrow 328) to the location shown. A plurality of dashed-line arrows have been provided here and in several of the below-described figures, to indicate a possible bloodflow direction through the blood vessel 324.

The isolation device 102, shown in an engaged position in FIG. 3, is held to the blood vessel wall 322 by the vacuum ports 118. The isolation rim 106, or another portion of the isolation device 102, contacts at least a portion of the blood vessel wall 322 longitudinally aligned with, and radially spaced from (as indicated by radial arrow 330), the anastomosis site 326. This relationship is further illustrated in the schematic cross-sectional view of FIG. 4, in which the radial direction coincides with the plane of the drawing page.

Optionally, the vacuum ports 118 apply vacuum or tension force to a portion of the blood vessel wall 322 substantially surrounding the anastomosis site 326. This exerted vacuum force helps to hold the isolation device 102 in place during formation of the anastomosis. It may be desirable, in certain applications of the present invention, to avoid the application of vacuum force directly to the anastomosis site 326.

Though not described further herein, it is also contemplated that one or more vacuum ports 118, on the isolation rim 106 or elsewhere on the apparatus 100, could be used to remove fluids or other materials from the blood vessel 324 or anastomosis site 326, instead of or in addition to exerting force to maintain the position of the isolation device 102. For example, once the isolation device 102 is held securely to the blood vessel wall 322, a vacuum port 118 or other feature of the apparatus 100 could be used to “clear” a working space 332 (defined between the working surface 104 and the blood vessel wall 322) through evacuation of blood from, and/or provision of saline or another fluid to, the working space. Optionally, this may be done in conjunction with one or more surgical tools (not shown) working from outside the blood vessel 324 at the anastomosis site 326.

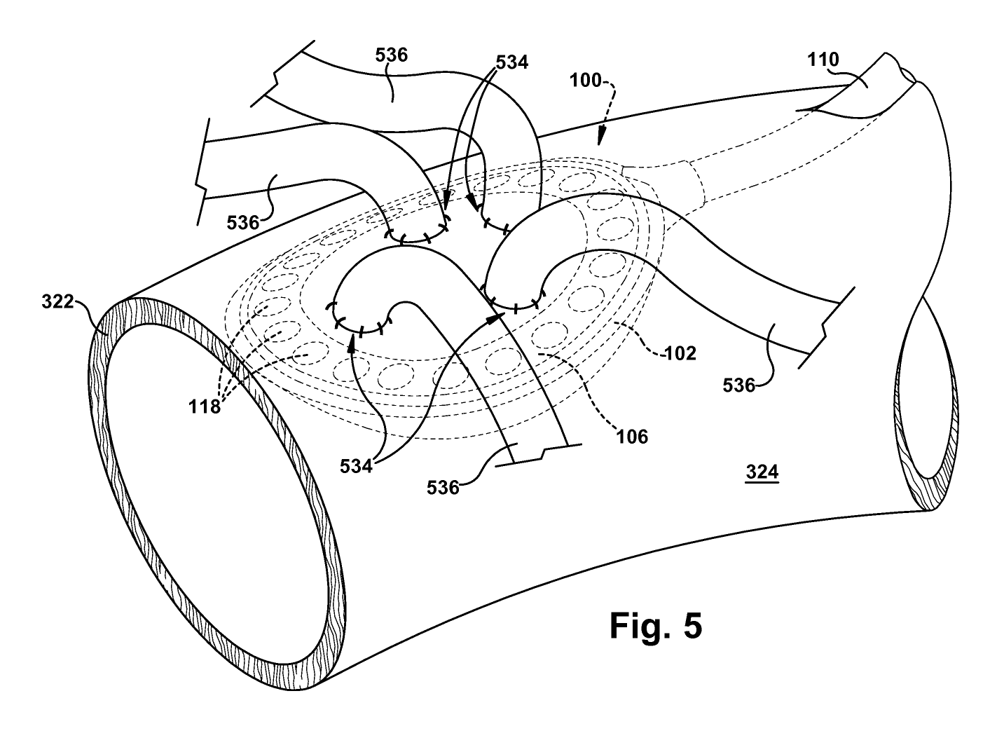

As shown in FIG. 5, one or more anastomoses 534 of graft vessels 536 could be performed within an area bounded inside the blood vessel 324 by the isolation rim 106. The anastomoses 534 shown here are of a traditional sutured type, but could also or instead be performed using any suitable anastomosis technique or device. Since a surgeon or other user will probably seek to avoid contact with the isolation rim 106 and other components of the apparatus 100 during the formation of these anastomoses, the isolation device 102 could include at least one indicator (not shown) to alert the surgeon of the position of the isolation device 102 within the blood vessel 324 from outside, such as, but not limited to, a visible or infrared light source, a tactile feature, a radiopaque marker, a magnetic or electric device (possibly linked to one or more surgical tools), a sensor/indicator system, or the like. Similarly, a separately provided shield (not shown) or toughened area of an isolation device 102 component could help prevent damage to the apparatus 100, should a scalpel or other surgical tool come into close proximity to the isolation device.

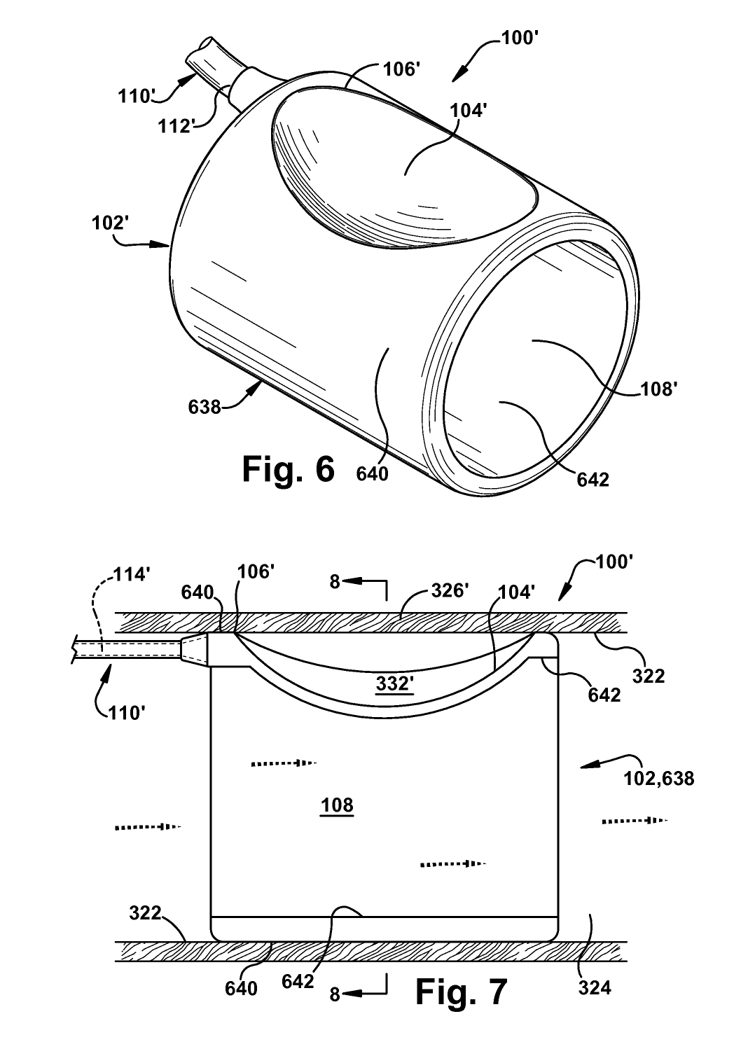

FIGS. 6-11 depict an apparatus 100′ according to a second embodiment of the present invention. The apparatus 100′ of FIGS. 6-11 is similar to the apparatus of FIGS. 1-5 and therefore, structures of FIGS. 6-11 that are the same as or similar to those described with reference to FIGS. 1-5 have the same reference numbers with the addition of a prime. Description of common elements and operation similar to those in the previously described first embodiment will not be repeated with respect to the second embodiment.

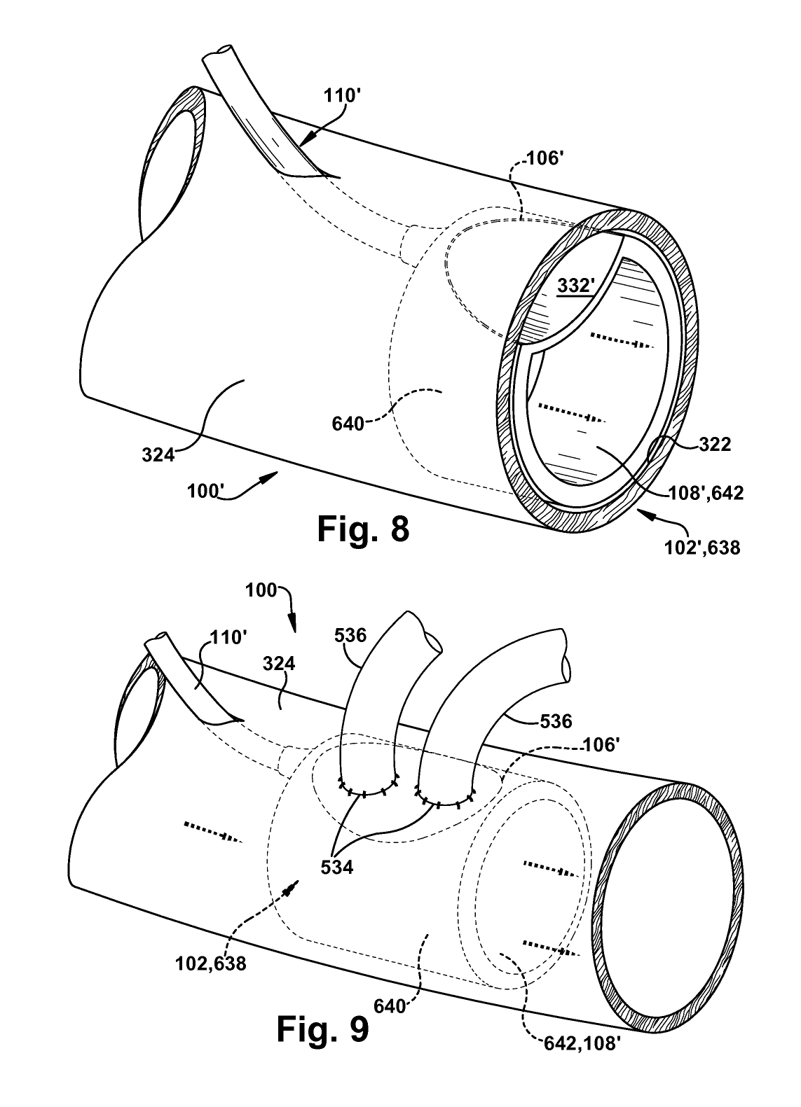

In accordance with the second example embodiment of the present invention, FIG. 6 depicts an isolation device 102′ including a concave working surface 104′ bounded by an isolation rim 106′ and a bloodflow surface 108′ opposite the working surface. In the second embodiment, a working space 332′ is formed between the working surface 104′ and the blood vessel wall 322, as shown in the cross-sectional view of FIGS. 7 and 8. However, unlike the tension/vacuum force of the first embodiment, the isolation device 102′ of the second embodiment exerts a compressive, outwardly-directed force against the blood vessel wall 322 to engage the blood vessel wall with the isolation device.

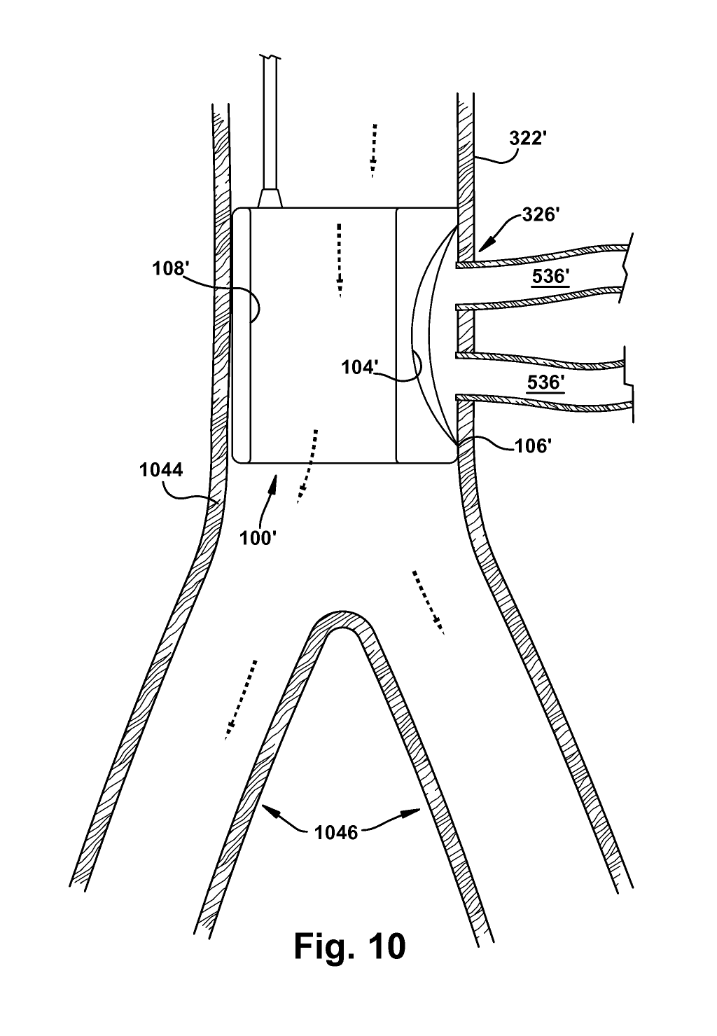

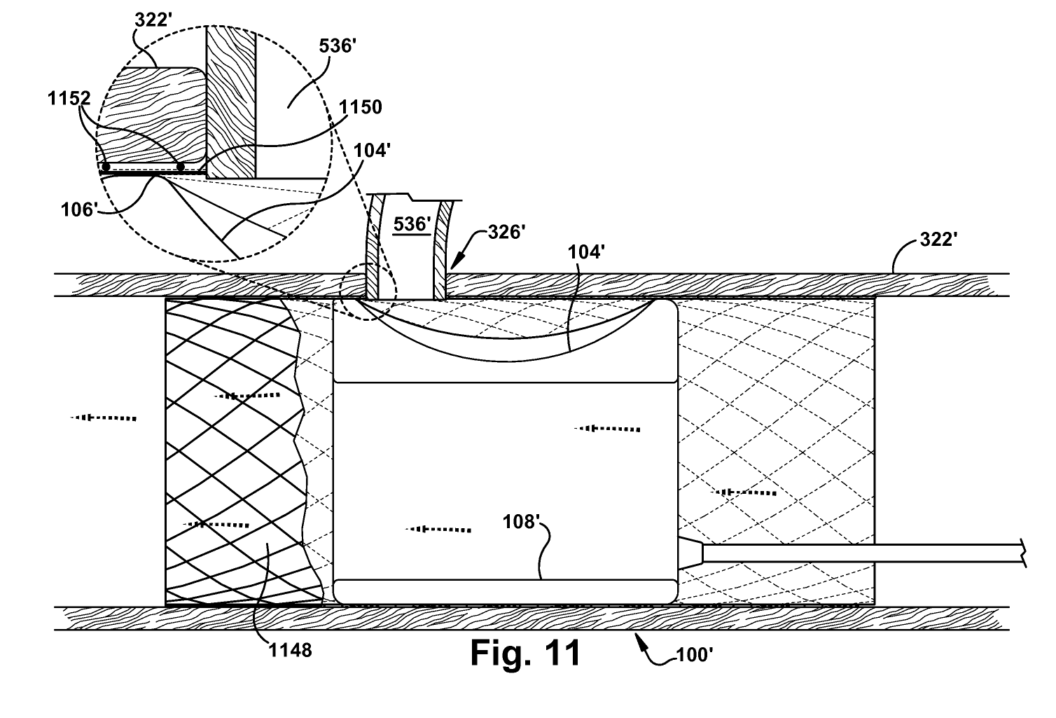

The isolation device 102′ of the second embodiment includes a balloon 638 having an outer balloon surface 640 configured to contact at least a portion of the blood vessel wall 322 longitudinally aligned with, and radially spaced from, the anastomosis site 326. The balloon 638 is depicted as tubular, but may be any shape. An inner balloon surface 642 defines the bloodflow surface 108′, along/through which bloodflow can continue traveling downstream from the anastomosis site. The inner balloon surface 642 may either echo the contours of the working surface 104′, as shown in FIGS. 6-8, or instead could be relatively uncontoured and substantially cylindrical, as shown in FIGS. 10-11. The tubular balloon 638 is configured for inflation by an inflation fluid source (not shown) via the operative lumen 114′ of the catheter 110′. For example, saline, air, or another suitable inflation fluid or combination thereof could be provided.

The outer balloon surface 640 is substantially contiguous with the (recessed) working surface 104′ and defines the isolation rim 106′ at the intersection of these two surfaces. The tubular balloon 638 exerts an outward compression force on at least a portion of the blood vessel wall 322 to at least partially engage the blood vessel wall 322 with the isolation device 102′.

As shown in FIG. 8, the anastomosis system has been introduced to the blood vessel 324 through a puncture in the blood vessel spaced apart from the anastomosis site 326 and advanced a relatively short distance to the anastomosis site when compared to the remotely-inserted option previously discussed. There is, however, no inherent preference for either insertion option, or any other suitable insertion technique, for use with any embodiment of the present invention in any use environment.

FIG. 9 depicts a side view of the apparatus 100′ in a first example use environment, with a plurality of graft vessels 536 attached to the blood vessel 324. As with the first embodiment, one of ordinary skill in the art can provide features (not shown) to the apparatus 100′ which may help in locating, securing, and/or protecting the components of the apparatus 100′ within the blood vessel to create the desired working space 332′.

FIG. 10 depicts a cross-sectional view of the apparatus 100′ in a second example use environment, within an aorta 1044. The apparatus 100′ in FIG. 10 is positioned just upstream of the branching of the iliac arteries 1046.

FIG. 11 depicts a partial cross-sectional (cutaway) view of the apparatus 100′ in a third example use environment, within a stent or graft 1148. The apparatus 100′ is located within graft 1148 inside the blood vessel 324′, and a graft vessel 536′ is being attached to the graft 1148. As can be seen in the magnified detail “A”, the isolation rim 106′ of the apparatus 100′ is in contact with a tissue lining 1150 of the graft 1148, although the tissue lining is an optional feature of the graft. When the tissue lining 1150 is absent, the isolation rim 106′ may be in direct contact with the mesh 1152 forming the graft 1148. As shown in detail “A”, however, the graft vessel 536′ passes through the blood vessel wall 322′ and the mesh 1152 at the anastomosis site 326′ and is anastomosed to the tissue lining 1150. It is also contemplated that the graft vessel 536′ could be attached to one or more of the blood vessel wall 322′, the mesh 1152, and the tissue lining 1150, and one of ordinary skill in the art can readily choose and create such attachments for a particular embodiment of the present invention.

It should be noted that, though the isolation device 102′ of the second embodiment is depicted as an inflatable tubular balloon 638, the isolation device 102′ could instead be a non-inflatable structure, such as a cuff device (not shown). For example, one of ordinary skill in the art could readily provide a pliable foam structure having dimensions similar to those of the depicted tubular balloon 638, but which would not require connection to an inflation source for deployment. When such a cuff device, made of any suitable material and in any suitable manner and configuration, is provided, the cuff includes an outer cuff surface configured to contact at least a portion of the vessel wall longitudinally aligned with, and radially spaced from, the anastomosis site. An inner cuff surface defines the bloodflow surface. The cuff device is configured for deployment from the insertion catheter. The outer cuff surface is substantially contiguous with the working surface and defines the isolation rim. The cuff device exerts an outward compression force on at least a portion of the blood vessel wall to at least partially engage the blood vessel wall with the isolation device.

Regardless of whether a cuff device or balloon 638 design is used, the working space 332′ is created in the second embodiment very similarly to the working space 332 created in the first embodiment. In both, a small space having substantially no bloodflow therein is isolated, both longitudinally and radially, from a continuing bloodflow in the radially and longitudinally adjacent portions of the blood vessel. The present invention provides for a relatively small portion of the interior volume of the blood vessel (namely, that portion directly adjoining the anastomosis site) to be held quiescent. Hence, bloodflow through the blood vessel is maintained, formation of thromboses is substantially avoided, blood loss is minimized, and downstream perfusion is maintained to avoid tissue ischemia.

When a surgeon or other user desires to form an anastomosis junction between a native blood vessel 324 and a graft vessel 536, the apparatus 100, 100′ is inserted into the patient in a desired manner at a location spaced apart from the anastomosis site 326, and is advanced to a location at or near the anastomosis site. Once in place as desired, the apparatus 100, 100′ is deployed into the blood vessel 324, with the deployment action depending upon the structure and configuration of the apparatus. For example, the isolation device 102, 102′ could be inflated, unfurled, decompressed, unrestrained, or otherwise deployed. When the apparatus 100, 100′ is fully deployed and in place at the anastomosis site 326, the isolation rim 106, 106′ is positioned in contact with at least a portion of the blood vessel 324 longitudinally aligned with, and radially spaced from, the anastomosis site. Optionally, the isolation rim 106, 106′ substantially surrounds the anastomosis site 326 in fairly close proximity along the blood vessel wall 322.

A force is then exerted on the blood vessel wall 322 to at least partially engage the blood vessel wall with the isolation device 102, 102′. The nature of that force (for example, tension or compression) depends upon the design of the apparatus 100, 100′ being used. The working space 332, 332′ is thereby formed and may be evacuated of blood or other matter as desired while bloodflow through the blood vessel 324 is maintained to avoid the deleterious effects on the patient which can result from a discontinued bloodflow.

The surgeon then accesses the working space 332, 332′ from outside the blood vessel 324. In this relatively clear and non-chaotic operating field, the surgeon can form one or more anastomoses 534 using one or more synthetic or natural tissue graft vessels 536, in any desired manner.

Once the desired type and number of anastomoses 534 have been formed, any deployment steps may be reversed to collapse the apparatus 100, 100′ for removal from the blood vessel 324. For example, the apparatus 100, 100′ could be reconstrained into the catheter 110, 110′ and/or any other cannula, trocar, catheter, or other insertion or removal assistance device used in a particular application of the present invention. Alternately, the apparatus 100, 100′ may be removed without collapse, via deconstruction or in any other suitable manner. The surgeon is then free to continue with the surgical procedure for which the anastomoses were made.

While aspects of the present invention have been particularly shown and described with reference to the preferred embodiment above, it will be understood by those of ordinary skill in the art that various additional embodiments may be contemplated without departing from the spirit and scope of the present invention. For example, any of the described structures and components could be made of any suitable material or combinations of materials; however, the chosen material(s) should be biocompatible for most applications of the present invention. Though certain components described herein are shown as having specific geometric shapes (e.g., the oval isolation rim 106 of the first embodiment), all structures of the present invention may have any suitable shapes, sizes, configurations, relative relationships, cross-sectional areas, or any other characteristics as desirable for a particular application of the present invention. For example, though shown as having certain configurations for the sake of description, the isolation devices could be any suitable shape. The isolation device could be configured to collapse and deploy in any suitable manner. The isolation device could have a structure, or portions thereof, with inflation/expansion, pliability, rigidity, solidity, hollowness, or any other physical properties as desired for a particular application of the present invention. The isolation device may be placed at the anastomosis site in any suitable manner, and need not be secured to a distal catheter end for insertion; for example, the isolation device could be advanced through a previously installed sheath, trocar, or tube which terminates near the anastomosis site. User access to the working space from the inside of the blood vessel may be provided via a lumen extending through the isolation device, or in any other suitable manner and via any structure of the apparatus, as desired. A device or method incorporating any of these features should be understood to fall under the scope of the present invention as determined based upon the claims below and any equivalents thereof.

Other aspects, objects, and advantages of the present invention can be obtained from a study of the drawings, the disclosure, and the appended claims.

Having described the invention, I claim:

1. An apparatus for fluidly isolating a portion of a blood vessel wall from bloodflow at an anastomosis site within a blood vessel, the apparatus comprising:
an insertion catheter having longitudinally spaced proximal and distal catheter ends and an operative lumen extending therebetween, and being configured for insertion into the blood vessel at an insertion location spaced apart from the anastomosis site;
an isolation device attached to the distal catheter end, including a concave working surface bounded by an isolation rim and a bloodflow surface opposite the working surface, the isolation rim being configured to contact at least a portion of the blood vessel longitudinally aligned with, and radially spaced from, the anastomosis site and the bloodflow surface being in contact with bloodflow past the anastomosis site within the blood vessel when the isolation device engages the blood vessel wall; and
retention means attached to the isolation device and in fluid communication with the operative lumen, the retention means being configured to exert force on the blood vessel wall to at least partially engage the blood vessel wall with the isolation device.

2. The apparatus of claim 1, wherein the retention means includes at least one vacuum port located on the isolation rim, the at least one vacuum port being in fluid connection with a vacuum source via the operative lumen, and the at least one vacuum port exerting an inward tension force to at least partially engage the blood vessel wall with the isolation device.

3. The apparatus of claim 1, wherein the retention means includes a tubular balloon having an outer balloon surface configured to contact at least a portion of the blood vessel wall longitudinally aligned with, and radially spaced from, the anastomosis site, an inner balloon surface defining the bloodflow surface, and being configured for inflation by an inflation fluid source via the operative lumen, the outer balloon surface being substantially contiguous with the working surface and defining the isolation rim, and the tubular balloon exerting an outward compression force on at least a portion of the blood vessel wall to at least partially engage the blood vessel wall with the isolation device.

4. The apparatus of claim 1, wherein the retention means includes a cuff device having an outer cuff surface configured to contact at least a portion of the blood vessel wall longitudinally aligned with, and radially spaced from, the anastomosis site, an inner cuff surface defining the bloodflow surface, and being configured for deployment from the insertion catheter, the outer cuff surface being substantially contiguous with the working surface and defining the isolation rim, and the cuff device exerting an outward compression force on at least a portion of the blood vessel wall to at least partially engage the blood vessel wall with the isolation device.

5. The apparatus of claim 1, wherein at least a portion of the isolation device is selectively collapsible for at least one of insertion to and removal from the blood vessel.

6. The apparatus of claim 1, wherein an insertion site of the insertion catheter is chosen responsive to a desired stability property of the isolation device.

7. An anastomosis system, comprising:
means for fluidly isolating an anastomosis site on a blood vessel wall from bloodflow through the blood vessel;
means for inserting the means for fluidly isolating into the blood vessel at a location spaced apart from the anastomosis site; and
means for retaining the means for fluidly isolating at the anastomosis site;
wherein, when the means for retaining is engaged, the means for fluidly isolating contacts at least a portion of the blood vessel longitudinally aligned with, and radially spaced from, the anastomosis site.

8. The anastomosis system of claim 7, wherein the means for retaining includes a means for applying vacuum force to a portion of the blood vessel wall substantially surrounding the anastomosis site.

9. The anastomosis system of claim 8, wherein the means for applying vacuum force avoids the application of vacuum force to the anastomosis site.

10. The anastomosis system of claim 7, wherein the means for retaining includes at least one of a tubular balloon and a cuff device, the means for retaining exerting an outward force upon at least a portion of a blood vessel diameter longitudinally aligned with the anastomosis site, and the at least one of the tubular balloon and the cuff device including a working surface spaced apart from the blood vessel wall at the anastomosis site and an inner surface allowing bloodflow therepast through the blood vessel.

11. The anastomosis system of claim 7, wherein at least a portion of the means for fluidly isolating is collapsible for at least one of insertion to and removal from the blood vessel.

12. A method for fluidly isolating a portion of a blood vessel wall from bloodflow at an anastomosis site within a blood vessel, the method comprising the steps of:
providing an insertion catheter having longitudinally spaced proximal and distal catheter ends and an operative lumen extending therebetween;
providing an isolation device attached to the distal catheter end, the isolation device including a concave working surface bounded by an isolation rim and a bloodflow surface opposite the working surface;
inserting the distal catheter end into the blood vessel at an insertion location spaced apart from the anastomosis site;
contacting at least a portion of the blood vessel longitudinally aligned with, and radially spaced from, the anastomosis site with the isolation rim;
contacting the bloodflow surface with bloodflow past the anastomosis site within the blood vessel; and
exerting force on the blood vessel wall to at least partially engage the blood vessel wall with the isolation device.

13. The method of claim 12, wherein the step of exerting force on the blood vessel wall includes the steps of:
providing at least one vacuum port on the isolation rim;
placing the at least one vacuum port in fluid communication with a vacuum source via the operative lumen; and
exerting an inward tension force to at least partially engage the blood vessel wall with the isolation device.

14. The method of claim 12, wherein the step of exerting force on the blood vessel wall includes the steps of:
providing a tubular balloon having an outer balloon surface, the outer balloon surface being substantially contiguous with the working surface and defining the isolation rim, and an inner balloon surface defining the bloodflow surface;
inflating the tubular balloon with an inflation source via the operative lumen;
contacting at least a portion of the blood vessel wall longitudinally aligned with, and radially spaced from, the anastomosis site with the outer balloon surface; and
exerting an outward compression force on at least a portion of the blood vessel wall with the tubular balloon to at least partially engage the blood vessel wall with the isolation device.

15. The method of claim 12, wherein the step of exerting force on the blood vessel wall includes the steps of:
providing a cuff device having an outer cuff surface, the outer cuff surface being substantially contiguous with the working surface and defining the isolation rim, and an inner cuff surface defining the bloodflow surface;
deploying the cuff device from the insertion catheter;
contacting at least a portion of the blood vessel wall longitudinally aligned with, and radially spaced from, the anastomosis site with the outer cuff surface; and
exerting an outward compression force on at least a portion of the blood vessel wall with the cuff device to at least partially engage the blood vessel wall with the isolation device.

16. The method of claim 12, including the step of selectively collapsing at least a portion of the isolation device for at least one of insertion to and removal from the blood vessel.
